(19) United States
(12) Patent Application Publication
(10) Pub. No.: US 2022/0387551 A1
(43) Pub. Date: Dec. 8, 2022

(54) METHODS USING SONICATION TO INCREASE BIOACTIVITY OF SOY PEPTIDES

(71) Applicant: SL TECHNOLOGY, INC., CHESTERFIELD, MD (US)

(72) Inventor: ALFREDO FLORES GALVEZ, FOLSOM, CA (US)

(73) Assignee: SL TECHNOLOGY, INC., CHESTERFIELD, MO (US)

(21) Appl. No.: 17/770,283

(22) PCT Filed: Oct. 20, 2020
(86) PCT No.: PCT/US2020/056398

§ 371 Date: Apr. 19, 2022

Related U.S. Application Data
(60) Provisional application No. 62/923,601, filed on Oct. 20, 2019.

(51) Int. Cl. A61K 38/16; A61K 9/08; A61K 41/00; A61K 36/48

(52) U.S. Cl. CPC A61K 38/168; A61K 9/08; A61K 41/0028; A61K 36/48

(57) ABSTRACT

This invention relates generally to methods for increasing bioactivity of lunasin in liquid formulations, sonicated bioactive lunasin compositions and methods of using sonicated bioactive lunasin. More specifically, this invention relates to methods of increasing bioactivity of lunasin in liquid formulations by administering sonication. One embodiment the present invention provides a method of increasing the bioactivity of lunasin comprising: a) providing a composition comprising lunasin, b) putting said composition into solution, and c) administering sonication to the composition.

### FIELD OF THE INVENTION

This invention relates generally to methods for increasing bioactivity of lunasin in liquid formulations, sonicated bioactive lunasin compositions made via such methods and methods of using sonicated bioactive lunasin. More specifically, this invention relates to methods of increasing bioactivity of lunasin in liquid formulations by administering sonication.

### BACKGROUND OF THE INVENTION

The soy peptide lunasin has been found to have a number of useful health applications. It has shown ability to inhibit histone acetylase enzyme P300/CBP-Associated Factor (PCAF) from acetylating histone H3, reduce expression of HMG CoA reductase and increase low density lipoprotein (LDL) receptor expression in mammals (U.S. Pat. No. 7,735,995). By inhibiting PCAF acetylation of histone H3, lunasin reduces the expression of HMG-Co A reductase in liver cells upon SREBP activation. (Bennet and Osborn PNAS (2000) 97:6340-6344) and lowers the expression of NFkB-regulated inflammation genes. (Pons et al. European Heart Journal (2009) 30: 266-277, Lu et al. Redox Biology (2019) 24:101221.) Lunasin specifically inhibits the acetylation of histone H3-Lysine 14, the key epigenetic biomarker associated with chronic inflammation, (Lu et al. Redox Biology (2019) 24:101221), atherosclerosis and heart disease formation, (Lu et al. Redox Biology (2019) 24:101221), and breast cancer formation (Wu et al. Nature Communications (2019) 10:1915). Lunasin has also been shown to inhibit expression of genes involved in cell proliferation and tumor formation, and increase expression of genes associated with tumor suppression, apoptosis, mitotic control and DNA repair (Galvez et al. Nutrition and Cancer (2011) 63: 623-636).

Lunasin has application for heart health in general and reducing cholesterol levels in specific (U.S. Pat. No. 9,814,757). Extensive scientific studies have shown that lunasin possesses inherent antioxidative (Garcia-Nebot M J, et al., Food Chem Toxicol. 2014 March; 65:155-61), anti-inflammatory (de Mejia E G, Dia V P., Peptides. 2009 December; 30(12):2388-98), and anticancerous (Galvez, A. F. et al., Cancer Res. 61:7473-7478 (2001) properties. Lunasin has applications in skin health (Shidal C, et al., Oncotarget. 2017 Apr. 11; 8(15):25525-25541). Lunasin has been shown to reduce skin tumor formation in mice when applied topically using alcohol-based skin delivery system. (Galvez, A. F., et al., Cancer Res. 61:7473-7478 (2001) and liposome delivery system (SBIR grant 1R43CA097690-01 https://www.sbir.gov/sbirsearch/detail/166681). It has applications for combating obesity (Chia-Chien Hsieh, et al., PLoS ONE 12(2): e0171969.) Lunasin has been shown to increase histone H4-Lysine 16 acetylation (Galvez et al. Nutrition and Cancer (2011) 63: 623-636), the key epigenetic biomarker linked to improvement in Alzheimer's patients and abnormally aging brains (Nativio et al. Nature Neuroscience (2018) 21: 497-505), Lunasin is currently used commercially as a dietary supplement.

When introduced orally, lunasin containing compositions, where lunasin is in complex with other proteins, are modified in the digestive process, thereby exposing bioactivity of the lunasin. However, where treatment with compositions containing lunasin will be via a route other than oral, the lunasin containing material must be released from the protein complex in order to expose the active portions of the lunasin peptide prior to its introduction via, for example, ophthalmic, topical or transdermal, nasal, inhalant, sublingual, buccal, ear, rectal, vaginal, or injection (e.g., intravenously, subcutaneously, intratumorally, intraperitoneally).

Lunasin is found in nature in complex with other proteins in the low-molecular weight albumin fraction of soy protein. The lunasin peptide contains two cysteine residues, one in the amino end and the other at the bioactive carboxyl end. These cysteine residues are believed to form disulfide linkages with other low molecular weight albumin proteins including Bowman-Birk trypsin inhibitor (BBIC), Kunitz trypsin inhibitor and the large subunit of methionine-rich 2s albumin (Gm2S-1) (de Souza et al. 2020 Biochimica et Biologica Acta 1868(8): 140440). It is believed that the ability of lunasin to complex with other proteins provides a cocoon conferring protection to the bioactive portions of lunasin from complete inactivation and digestion of the peptide when taken orally. Extraction processes have been developed to preserve these protein complexes and produce lunasin enriched soy powders that protect most of the lunasin from digestion and resultant inactivation, while retaining lunasin bioactivity when ingested.

Because lunasin is often found in complex with other proteins, when the lunasin enriched soy powders are dissolved in buffered liquid solutions to extract soluble proteins, the bioactivity of lunasin is lower than desired. Without being bound to any particular mechanism of action, it is believed that the protein complexes that protect the lunasin bioactive site from complete digestion and resultant inactivation also block the bioactive portions of the lunasin peptide that need to be exposed for the desired bioactivity to be present, thereby limiting the amount of bioactive lunasin in the solution. The bioactivity of lunasin enriched soy powders is increased when it passes through the gastro-intestinal tract, because the digestion process releases bioactive lunasin from protein complexes before being absorbed into the body. In order to treat a person with lunasin enriched soy via routes other than oral, such as sublingual, topical, or inhalation routes, lunasin needs to be released from its protein complex cocoon to make it more bioactive. For treatment routes other than oral, ex vivo treatment of lunasin enriched soy powder with proteases has been shown to increase the bioactivity of the lunasin contained therein (U.S. Pat. No. 8,598,111). However, digestion is time consuming, because it involves several steps. It requires additional chemicals which can be expensive to scale up in a commercial setting. Food grade enzymes, which are even more expensive and/or hard to obtain, are required for certain administrations, such as sublingual. It can also be hard to control the extent of digestion that occurs, which complicates efforts to maximize bioactivity of treated lunasin.

Accordingly, there exists a need for improved methods of increasing bioactivity in lunasin for treatment routes other than oral, improved compositions and methods of using compositions created using those methods. The present invention provides these and other related benefits.

### Definitions

To facilitate an understanding of the invention, a number of terms and phrases are defined below. Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The general techniques and procedures described or referenced herein are generally well understood and commonly employed using conventional methodology by those skilled in the art. As appropriate, procedures involving the use of commercially available kits and reagents are generally carried out in accordance with manufacturer defined protocols and/or parameters unless otherwise noted.

As used herein, the singular form “a”, “an”, and “the” includes plural references unless indicated otherwise. For example, “a” protease enzyme inhibitor includes one or more protease enzyme inhibitors.

As used herein “ug” is an abbreviation for microgram, “uL” in an abbreviation for microliter, and “uM” is an abbreviation for micromole.

As used herein, “biological activity” and “bioactivity” refer to the in vivo activities of a compound or physiological responses that result upon in vivo administration of a compound, composition, or other mixture. Biological activity and bioactivity, thus, encompass therapeutic effects and pharmaceutical activity of such compounds, compositions and mixtures. Biological activities and bioactivities may be observed and measured in in vitro systems designed to test or use such activities also.

As used herein, the terms “biologically active” and “bioactive” refers to a molecule having structural, regulatory and or biochemical functions of a naturally occurring molecule.

As used herein, the term “sonicated lunasin” refers to lunasin that has been treated with sonication while in solution.

As used herein, the term “sonicated bioactive lunasin” refers to lunasin that has been treated with sonication in solution and retains structural, regulatory and or biochemical functions of a naturally occurring lunasin molecule while showing increased ability to prevent the acetylation of histone H3 by the PCAF enzyme following sonication. The ability to prevent the acetylation of histone H3 by the PCAF enzyme can be measured using the assay set forth in Example 2, below.

As used herein, a “combination” refers to any association between two or among more items.

As used herein, the terms “disease” “disorder” and “pathological condition” are used interchangeably to describe a state, signs, and/or symptoms that are associated with any impairment of the normal state of a living animal or of any of its organs or tissues that interrupts or modifies the performance of normal functions, and may be a response to environmental factors (such as malnutrition, industrial hazards, or climate), to specific infective agents (such as worms, bacteria, or viruses), to inherent defect of the organism (such as various genetic anomalies, or to combinations of these and other factors.

As used herein, the term “effective amount” refers to the amount of a composition (e.g., comprising lunasin) sufficient to effect beneficial or desired results. An effective amount can be administered in one or more administrations, applications or dosages and is not intended to be limited to a particular formulation or administration route.

As used herein, the terms “administration” and “administering” refer to the act of giving a drug, prodrug, or other agent, or therapeutic treatment (e.g., compositions of the present invention) to a subject (e.g., a subject or in vivo, in vitro, or ex vivo cells, tissues, and organs) and/or to direct, instruct, or advise the use of the composition for any purpose (preferably, for a purpose described herein). Where the administration of one or more of the present compositions is directed, instructed or advised, such direction may be that which instructs and/or informs the user that use of the composition may and/or will provide one or more of the benefits described herein.

Exemplary routes of administration to the human body can be through the eyes (ophthalmic), mouth (oral), skin (topical or transdermal), nose (nasal), lungs (inhalant), sublingual or oral mucosa (buccal), ear, rectal, vaginal, by injection (e.g., intravenously, subcutaneously, intratumorally, intraperitoneally, etc.) and the like.

Administration which is directed may comprise, for example, oral direction (e.g., through oral instruction from, for example, a physician, health professional, sales professional or organization, and/or radio or television media (i.e., advertisement) or written direction (e.g., through written direction from, for example, a physician or other health professional (e.g., scripts), sales professional or organization (e.g., through, for example, marketing brochures, pamphlets, or other instructive paraphernalia), written media (e.g., internet, electronic mail, or other computer-related media), and/or packaging associated with the composition (e.g., a label present on a package containing the composition). As used herein, “written” includes through words, pictures, symbols, and/or other visible descriptors. Such direction need not utilize the actual words used herein, but rather use of words, pictures, symbols, and the like conveying the same or similar meaning are contemplated within the scope of this invention.

As used herein, the term “treatment” or grammatical equivalents encompasses the improvement and/or reversal of the symptoms of disease (e.g., heart disease). A composition which causes an improvement in any parameter associated with disease when used in the screening methods of the instant invention may thereby be identified as a therapeutic composition. The term “treatment” refers to both therapeutic treatment and prophylactic or preventative measures. For example, those who may benefit from treatment with compositions and methods of the present invention include those already with a disease and/or disorder (e.g., elevated cholesterol levels) as well as those in which a disease and/or disorder is to be prevented (e.g., using a prophylactic treatment of the present invention).

As used herein, the terms “individual,” “subject” and “patient” refer to any animal, including but not limited to, human and non-human animals (for example, without limitation, primates, dogs, cats, cows, horses, sheep, rodents, poultry, fish, crustaceans, etc.) that is studied, analyzed, tested, diagnosed or treated. As used herein, the terms “individual,” “subject” and “patient” are used interchangeably, unless indicated otherwise.

As used herein, the term “antibody” (or “antibodies”) refers to any immunoglobulin that binds specifically to an antigenic determinant, and specifically binds to proteins identical or structurally related to the antigenic determinant that stimulated their production. Thus, antibodies can be useful in assays to detect the antigen that stimulated their production. Monoclonal antibodies are derived from a single clone of B lymphocytes (i.e., B cells), and are generally homogeneous in structure and antigen specificity. Polyclonal antibodies originate from many different clones of antibody-producing cells, and thus are heterogeneous in their structure and epitope specificity, but all recognize the same antigen. Also, it is intended that the term “antibody” encompass any immunoglobulin (e.g., IgG, IgM, IgA, IgE, IgD, etc.) obtained from any source (e.g., humans, rodents, non-human primates, lagomorphs, caprines, bovines, equines, ovines, etc.).

As used herein, the term “antigen” is used in reference to any substance that is capable of being recognized by an antibody.

As used herein, the term “toxic” refers to any detrimental or harmful effects on a subject, a cell, or a tissue as compared to the same cell or tissue prior to the administration of the toxicant.

As used herein, the term “pharmaceutical composition” refers to the combination of an active agent (e.g., lunasin) with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “topically” refers to application of the compositions of the present invention to the surface of the skin and mucosal cells and tissues (e.g., alveolar, buccal, lingual, masticatory, or nasal mucosa, and other tissues and cells that line hollow organs or body cavities).

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), liposomes and various types of wetting agents, any and all solvents, dispersion media, coatings, sodium lauryl sulfate, isotonic and absorption delaying agents, disintrigrants (e.g., potato starch or sodium starch glycolate), nanomolecules and the like. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. (1975), incorporated herein by reference).

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the terms “gene expression” and “expression” refer to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refer to regulation that increases and/or enhances the production of gene expression products (e.g., RNA or protein), while “down-regulation” or “repression” refer to regulation that decrease production. Molecules (e.g., transcription factors) that are involved in up-regulation or down-regulation are often called “activators” and “repressors,” respectively.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

As used herein amino acid refers to any of the naturally occurring amino acids having the standard designations listed in Table 1, below. It also refers to those known synthetic amino acids. Unless otherwise indicated, all amino acid sequences listed in this disclosure are listed in the order from the amino terminus to the carboxyl terminus. As used herein, the abbreviations for any protective groups, amino acids and other compounds, are in accord with their common usage, recognized abbreviations, or the IUPAC-IUB Commission on Biochemical Nomenclature, unless otherwise indicated (see Biochemistry 11: 1726 (1972)). As used herein, amino acid residues are represented by the full name thereof, by the three letter code corresponding thereto, or by the one-letter code corresponding thereto, as indicated in the following table:

TABLE 1

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

As used herein, the terms “peptide,” “polypeptide” and “protein” all refer to a primary sequence of amino acids that are joined by covalent “peptide linkages.” In general, a peptide consists of a few amino acids, typically from 2-50 amino acids. The term “polypeptide” encompasses peptides and proteins, wherein the term “protein” typically refers to large polypeptides and the term “peptide” typically refers to short polypeptides. In some embodiments, the peptide, polypeptide or protein is synthetic, while in other embodiments, the peptide, polypeptide or protein is recombinant or naturally occurring. A “synthetic” peptide is a peptide that is produced by artificial means in vitro (i.e., was not produced in vivo). The term “peptide” further includes modified amino acids (whether naturally or non-naturally occurring), such modifications including, but not limited to, phosphorylation, glycosylation, pegylation, lipidization and methylation.

As applied to polypeptides, the term “substantial identity” means that two peptide sequences, when optimally aligned, such as by the programs GAP or BESTFIT using default gap weights, share at least 80% sequence identity, preferably at least 90% sequence identity, more preferably at least 95% sequence identity or more (e.g., 99% sequence identity). Preferably, residue positions which are not identical differ by conservative amino acid substitutions.

The phrase “functionally equivalent” means that the variant, analogue or fragment of a lunasin polypeptide retains a desired bioactivity in common with the lunasin polypeptide. In at least one embodiment of the present invention, the desired bioactivity in common with lunasin is bioactivity related to the control, stabilization, or reduction in production or existing levels of cholesterol, LDL cholesterol, total cholesterol, or lipids. Preferably, a given quantity of the analogue, variant or fragment is at least 10%, preferably at least 30%, more preferably at least 50, 60, 80, 90, 95 or 99% as effective as an equivalent amount of the naturally occurring lunasin from which the analogue, variant or fragment is derived. Determination of the relative efficacy of the analogue, variant or fragment can readily be carried out by utilizing a prescribed amount of the analogue, variant or fragment in the one or more of the assay methods of the invention and then comparing the ability of the analogue, variant or fragment to naturally occurring lunasin in tests that measure the ability of the sample to inhibit the acetylation of histone H3, or to effect the expression of HMG Co-A reductase, Sp1 or LDL-receptor.

The term “analogue” as used herein with reference to a polypeptide means a polypeptide which is a derivative of the polypeptide of the invention, which derivative comprises addition, deletion, and/or substitution of one or more amino acids, such that the polypeptide retains substantially the same function as the lunasin polypeptide identified below.

The term “fragment” refers to a polypeptide molecule that is a constituent of the full-length lunasin polypeptide and possesses qualitative bioactivity in common with the full-length lunasin polypeptide. The fragment may be derived from the full-length lunasin polypeptide or alternatively may be synthesized by some other means, for example chemical synthesis. By reference to “fragments” it is intended to encompass fragments of a protein that are of at least 5, preferably at least 10, more preferably at least 20 and most preferably at least 30, 40 or 50 amino acids in length and which are functionally equivalent to the protein of which they are a fragment.

The term “variant” as used herein refers to a polypeptide which is produced from a nucleic acid encoding lunasin, but differs from the wild type lunasin in that it is processed differently such that it has an altered amino acid sequence. For example a variant may be produced by an alternative splicing pattern of the primary RNA transcript to that which produces wild type lunasin.

Analogues and variants are intended to encompass proteins having amino acid sequence differing from the protein from which they are derived by virtue of the addition, deletion or substitution of one or more amino acids to result in an amino acid sequence that is preferably at least 60%, more preferably at least 80%, particularly preferably at least 85, 90, 95, 98, 99 or 99.9% identical to the amino acid sequence of the original protein. The analogues or variants specifically include polymorphic variants and interspecies analogues. The analogues and variants of the present invention further may have “conservative” changes, wherein a substituted amino acid has similar structural or chemical properties. One type of conservative amino acid substitution refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. More rarely, a variant may have “non-conservative” changes (e.g., replacement of a glycine with a tryptophan). Similar minor variations may also include amino acid deletions or insertions (i.e., additions), or both. Guidance in determining which and how many amino acid residues may be substituted, inserted or deleted without abolishing bioactivity may be found using computer programs well known in the art, for example, DNAStar software. Variants can be tested in functional assays such as those described in the Examples section below.

As used herein “lunasin” refers to the natural soybean lunasin polypeptide set forth in (SEQ. ID. 2). Additional description of the Lunasin peptide and an evaluation of various functionally equivalent fragments and analogues appear in U.S. Pat. Nos. 6,107,287, 6,544,956, US Patent Application 2003/0229038, filed Nov. 22, 2002, U.S. Pat. No. 6,391,848, U.S. patent application Ser. No. 10/252,256, filed Sep. 23, 2002, and U.S. patent application Ser. No. 10/302,633, filed Nov. 22, 2002, all of which are hereby incorporated by reference herein in their entirety for all purposes. These disclosures will guide one skilled in the art in identifying functionally equivalent and biologically active fragments, variants and analogues of lunasin.

As used herein “lunasin enriched” refers to compositions containing biologically active levels of naturally occurring lunasin, or a naturally occurring analogue of lunasin, that is at a concentration greater than that at which lunasin is found in the material used as the source of that lunasin or analogue. As used herein “lunasin enriched soy powder” refers to powdered compositions containing biologically active levels of naturally occurring lunasin, or a naturally occurring analogue of lunasin, that is at a concentration at least twice than that at which lunasin is naturally found in the source seed. As used herein “lunasin enriched soy extract” refers to compositions extracted from soy containing biologically active levels of lunasin that is at a concentration at least twice that at which lunasin is naturally found in the source soy seed. Without limiting the invention to any particular source of the compositions of the present invention, lunasin enriched compositions can be obtained from soybean, soy isolates, soy concentrates, or other soy derived products, whether or not commercially obtained, and other sources of seed proteins such as those found in wheat, barley, amaranth and quinoa.

As used herein “lunasin protecting soy flour” refers to soy flour compositions comprising soy flour and an amount of a protease inhibitor sufficient to protect lunasin, or an analogue, variant or fragment thereof, from complete digestion, wherein the compositions are extracted by mechanical means without the use of organic solvents and do not have levels of anti-nutritional elements that would cause an adverse effect in an individual who ingested them.

As used herein “digested” refers to the treatment of a polypeptide with a digestive material that breaks it down into its component amino acids. Examples of digestive materials that can be used are well known in the art, and include, without limitation, pancreatin and other proteases such as trypsin, chymotrypsin, pepsin, Proteinase K, thermolysin, thrombin, Arg-C proteinase, Asp-N endopeptidase, AspN endopeptidase+N-terminal Glu, BNPS-Skatole, CNBr, clostripain, formic acid, glutamyl endopeptidase, iodosobenzoic acid, LysC, LysN, NTCB (2-nitro-5-thiocyanobenzoic acid), and Staphylococcal peptidase.

As used herein “partially digested biologically active” in relation to a polypeptide refers to the treatment of a polypeptide with a digestive material under conditions that increase the bioactivity of the polypeptide.

Referenced herein are trade names for components including various ingredients utilized in the present invention. The inventors herein do not intend to be limited by materials under a certain trade name. Equivalent materials (e.g., those obtained from a different source under a different name or reference number) to those referenced by trade name may be substituted and utilized in the descriptions herein.

The compositions herein may comprise, consist essentially of, or consist of any of the elements as described herein.

The practice of the present invention will employ, unless otherwise indicated, conventional techniques of molecular biology (including recombinant techniques), microbiology, cell biology, biochemistry, immunology, and protein kinetics, which are within the skill of the art. Such techniques are explained fully in the literature. Furthermore, procedures employing commercially available assay kits and reagents will typically be used according to manufacturer-defined protocols unless otherwise noted.

### SUMMARY OF THE INVENTION

The present disclosure relates to methods for increasing bioactivity of lunasin in liquid formulations, compositions made via such methods and methods of using such compositions.

One embodiment the present invention provides a method of increasing the bioactivity of lunasin comprising: a) providing a composition comprising lunasin, b) putting said composition into solution, and c) administering sonication to the composition. Another embodiment of the present invention provides a method of increasing the bioactivity of lunasin, comprising: providing a composition comprising lunasin in solution and administering sonication to the composition at an amplitude and time interval that increases the bioactivity of lunasin.

In a specific embodiment of the above methods of the present invention, the methods further comprises, after administering sonication to the composition, testing the composition for bioactivity of said lunasin. In another specific embodiment of this method, sonication is administered at an amplitude of between 10 Watts and 900 Watts, or more specifically an amplitude of between 20 Watts and 500 Watts, or even more specifically at an amplitude of between 30 Watts and 400 Watts or, very specifically, at approximately 360 Watts.

In another specific embodiment of this method, sonication is administered for a period of between 1 and 20 minutes or, more specifically, for a period of between 2 and 10 minutes, or yet more specifically, for a period of approximately 4 minutes.

In another specific embodiment of this method, sonication is administered in pulses lasting between 1 and 5 seconds. In another specific embodiment of the present invention, the pulses lasting between 1 and 5 seconds are alternated with pauses lasting between 1 and 5 seconds.

In another specific embodiment of this method, the composition that is sonicated comprises lunasin enriched soy powder, or it comprises soy concentrate and soy flour.

In another specific embodiment of this method the composition is in solution at a concentration of from 0.5 mg/ml to 70 mg/ml, or, more specifically the composition is in solution at a concentration of from 5 mg/ml to 20 mg/ml, or, even more specifically, the composition is in solution at a concentration of approximately 10 mg/ml.

In another specific embodiment of this method, the composition is in an aqueous solution.

One embodiment the present invention encompasses a product made by providing a composition comprising lunasin in solution, and administering sonication to the composition at an amplitude and time interval that increases the bioactivity of lunasin.

One embodiment the present invention provides a composition produced by the method of providing a composition comprising lunasin; b) putting said composition into solution, and b) administering sonication to the composition. Another embodiment the present invention provides a composition produced by providing a composition comprising lunasin in solution and administering sonication to the composition at an amplitude and time interval that increases the bioactivity of lunasin.

A specific embodiment the present invention provides a composition produced by the method of providing a composition comprising lunasin, b) putting said composition into solution, c) administering sonication to the composition and d) testing the composition for bioactivity of said lunasin.

A specific embodiment the present invention provides a composition produced by the method of providing a composition comprising lunasin and b) putting said composition into solution, wherein the sonication is administered for approximately 4 minutes.

A specific embodiment the present invention provides a composition produced by the method of providing a composition comprising lunasin and b) putting said composition into solution, wherein the composition is in solution at a concentration of approximately 10 mg/ml.

Another embodiment of the present invention is a composition comprising sonicated bioactive lunasin.

In another embodiment, the present invention provides a method of treating a human with lunasin, comprising: (a) providing: (i) an individual desiring or needing treatment with lunasin, and (ii) a composition comprising sonicated bioactive lunasin; and (b) administering said composition to said subject. In specific embodiments the composition is administered topically, sublingually, nasally, transdermally, via injection, via the eye, or orally.

In another embodiment, the present invention provides a method treating a human with lunasin, comprising: (a) providing: (i) an individual desiring or needing treatment with lunasin, and (ii) a composition comprising lunasin in solution, b) administering sonication to the composition at an amplitude and time interval that increases the bioactivity of lunasin, and (c) administering said composition to said subject. In specific embodiments the composition is administered topically, sublingually, nasally, transdermally, via injection, via the eye or orally.

### DESCRIPTION OF THE FIGURES

The above-mentioned features and objects of the present disclosure will become more apparent with reference to the following description taken in conjunction with the accompanying drawings wherein like reference numerals denote like elements and in which:

FIG. 1 shows the 2S albumin protein encoded by Gm2S 1 cDNA (SEQ ID NO 1). Arrows indicate endoproteolytic sites that give rise to small subunit (lunasin) (SEQ ID NO 2) and the large subunit (methionine rich protein). Important regions in both subunits are indicated.

### GENERAL DESCRIPTION OF THE PREFERRED EMBODIMENTS

This invention relates generally to methods for increasing bioactivity of lunasin in liquid formulations, compositions made via such methods and methods of using such compositions. More specifically, this invention relates to methods of increasing bioactivity of lunasin in liquid formulations using sonication, compositions created using the sonication methods, and methods of using the sonicated compositions.

Lunasin

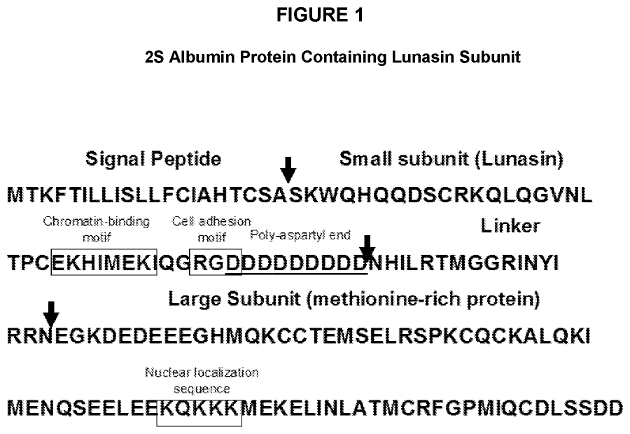

Lunasin is a bioactive component in soybean (Glycine max) with a novel chromatin-binding property and epigenetic effects on gene expression. Lunasin is the small subunit peptide of a cotyledon-specific 2S albumin. FIG. 1 shows the 2S albumin protein (SEQ ID NO 1) and the small lunasin subunit (SEQ ID NO 2). The lunasin soy peptide is heat stable, water soluble and found in significant amounts in select soy protein preparations, and significant guidance is provided in the literature on selection of sources of soy and soy products for isolation or concentration of lunasin.

Studies show that lunasin can enter mammalian epithelial cells through its RGD cell adhesion motif, bind preferentially to deacetylated histones and inhibit histone H3 and H4 acetylation. (Galvez, A. F., et al., Cancer Res. 61:7473-7478 (2001)). There is growing evidence that cellular transformation, responses to hormones and dietary and environmental effects involve epigenetic changes in gene expression, which are modulated by the reversible processes of DNA methylation-demethylation and histone acetylation-deacetylation. (De Pinho, R. A., Nature 391: 533-536 (1998), Kuzmin I. & Geil L. IntArch Biosci 2001: 1047-1 056 (2001)).

The bioactivity of lunasin is believed to be based on its ability to bind to histone H3 and prevent its acetylation by the PCAF enzyme. Lunasin is the first natural substance to be identified as a histone acetylase inhibitor, although it does not directly affect the histone acetylase enzyme. It is believed to inhibit H3 and H4 acetylation by binding to specific deacetylated lysine residues in the N-terminal tail of histones H3 and H4, making them unavailable as substrates for histone acetylation. The elucidation of the mechanism of action has made lunasin an important molecule for research studies to understand the role of epigenetics and chromatin modifications in important biological processes.

A study on the effect of lunasin on prostate carcinogenesis at the University of California at Davis revealed the effects of lunasin on histone H4 modifications and the up regulation of chemopreventive genes. (Magbanua M, Dawson K, et al., Nutritional Genomics—Discovering the Path to Personalized Nutrition, J. Kaput and R. L. Rodriguez eds., Wiley and Sons, New Jersey (2005)). Lunasin has been shown to bind and inhibit histone H4-Lysine 8 changing the conformation of the histone H4 tail to allow increased acetylation of histone H4-Lysine 16 (Galvez et al. Nutrition and Cancer (2011) 63: 623-636). The increase in H4-Lysine 16 acetylation provides a mechanism of action to explain how lunasin can upregulate expression of chemopreventive genes in prostate cells (U.S. Pat. No. 9,678,060).

The specific biological effect of lunasin binding to deacetylated histone H3 and inhibition of acetylation, the induction of genes involved in cholesterol biosynthesis by the sterol regulatory element binding proteins (SREBP) are set forth in U.S. Pat. No. 9,814,757, which teaches an in vitro histone acetyltransferase (HAT) assay to show that lunasin significantly inhibits histone H3 acetylation by the histone acetylase enzyme, PCAF.

The bioactivity of lunasin is further supported by large scale epidemiological and clinical data linking soy protein consumption with lower LDL cholesterol, and lower risk of cardiovascular disease. (Anderson J W, et al., N Eng J Med 333: 276-282 (1995)). The identification of lunasin as the main component in soy protein that confers its cholesterol-lowering property has paves the way for optimizing soy protein ingredients to maximize its heart-healthy benefits.

It has been shown that constitutive expression of the lunasin gene in mammalian cells disturbs kinetochore formation and disrupts mitosis, leading to cell death. When applied exogenously in mammalian cell culture, the lunasin peptide suppresses transformation of normal cells to cancerous foci that are induced by chemical carcinogens and oncogenes. Lunasin's chemopreventive mechanism of action has been shown by the fact that lunasin (a) is internalized through its RGD cell adhesion motif, (b) colocalizes with hypoacetylated chromatin in telomeres at prometaphase, (c) binds preferentially to deacetylated histone H4, which is facilitated by the presence of a structurally conserved helical motif found in other chromatin-binding proteins, (d) inhibits histone H3 and H4 acetylation, and (e) induces apoptosis in E1A-transfected cells. (Galvez, et al., Cancer Res. 61:7473-7478 (2001)). Based on these results, a novel chemopreventive mechanism has been proposed wherein lunasin gets inside the nucleus, binds to deacetylated histones, prevents their acetylation and inhibits gene expression like those controlled by the Rb tumor suppressor and h-ras oncogene.

Microarray experiments have demonstrated minimal to no negative genetic changes using lunasin and suggest that lunasin can act as a transcriptional activator of genes that protect normal cells from transformation. (Magbanua M, et al., J. Kaput and R. L. Rodriguez eds., Wiley and Sons, New Jersey (2005)).

Lunasin content in different soy varieties, soy protein concentrates and soy protein isolates vary significantly from one preparation to another. It appears that lunasin is the only bioactive agent from soy with a viable molecular mechanism of action that can explain the cholesterol-lowering property of soy protein. The data also helps elucidate the widely divergent clinical results seen in previous studies. Because lunasin is present in varying amounts in various soy protein preparations, it appears that the unpredictable results with respect to cholesterol lowering effects and the absence of dose dependent effects in previous studies using soy protein isolates, even those that tested higher concentrations of soy protein, is likely due to either variation in the amounts of lunasin or the absence of chymotrypsin inhibitors to protect the lunasin during digestion or a combination of the two factors.

Although the concentration and bioactivity of lunasin in various commercially available sources vary, among the different sources of lunasin, soy protein concentrates have shown higher yield of bioactive lunasin in some experiments. In a preferred embodiment of the present invention, soy concentrates, soy protein concentrates and/or soy concentrates combined with soy flour are sources of lunasin for use in compositions and methods of the present invention.

Sonication

Sonication is the practice of using sound waves to agitate particles in a solution. Sonication converts an electrical signal into a physical vibration to break substances apart. During sonication, cycles of pressure form thousands of microscopic vacuum bubbles in the solution. The bubbles collapse into the solution in a process known as cavitation. This causes powerful waves of vibration that release an enormous energy force in the cavitation field, which disrupts molecular interactions such as interactions between molecules of water, separates clumps of particles, and facilitates mixing. Ultrasonic frequencies are generally used.

Among the many applications, these disruptions can mix solutions, speed dissolution and break up aggregates of particles, provide energy to speed chemical reactions and degas liquids. In biological applications it can deactivate biological matter, disrupt cell membranes to release cell contents, such as proteins, for testing, and fragment DNA. It is useful for the extraction of compounds from solution. In the laboratory, it is usually applied using an ultrasonic bath or an ultrasonic probe. Sonication treatment is advantageous because it is simple, inexpensive and controllable.

Sonication is performed at a variety of amplitudes, and can be performed in one or a series of pulses with varying time intervals between treatments.

Sonication to Increase Lunasin Bioactivity.

Partial digestion of lunasin enhances or increases the bioactivity of lunasin in various soy compositions. (U.S. Pat. No. 8,598,111). However, digestion is time consuming. It requires several steps, additional chemicals and protease enzymes which are expensive to scale up in a commercial setting. Food grade enzymes, which are even more expensive and/or hard to obtain, are required for certain administrations, such as sublingual. It can also be hard to control the extent of digestion which complicates efforts to maximize bioactivity of treated lunasin. Slight changes in protocol, such as increasing incubation temperature and time can result in complete destruction of lunasin bioactivity.

Sonication is a rapid process because it does not involve additional digestion and enzymatic steps. It is inexpensive and easy to control. It can be performed with our without additional reagents, but no additional reagents are required. Sonication produces a random distribution of agitating energy, resulting in a mechanical process that is easier to control and optimize in order to get maximum lunasin bioactivity.

A new method is shown to release lunasin from protein complexes in compositions containing lunasin using mechanical shearing through sonication, in order to increase lunasin bioactivity. Without being bound to any particular mechanism of action, it is believed that Sonication of liquid compositions containing lunasin results in its release from protein complexes and the increase in bioavailability of active sites of the lunasin peptide.

Sonication is frequently used to mix substances that are not similarly soluble. Sonication can also break apart peptides. Too much sonication will destroy lunasin bioactivity. However, lunasin will retain bioactivity even if the peptide is fragmented to a certain extent during sonication. Lunasin retains bioactivity after fragmentation providing that at least a 15 amino acid fragment of lunasin that contains the amino acids from the entire chromatin binding motif through the first three aspartic acids remain intact.

In one embodiment of the present invention, parameters for sonication conditions to provide the most sonicated bioactive lunasin when sonicating lunasin containing compositions dissolved in buffered aqueous solutions or pure water (1:2 w/v) are provided as follows: sonicate at an amplitude of from 90 Watts to 500 Watts for from 2 to 5 minutes duration with short pulses and short pauses in between pulses. In a preferred embodiment, the pulses and pauses are for approximately 2 seconds each. In another embodiment of the present invention, parameters for sonication conditions to provide the most sonicated bioactive lunasin when sonicating lunasin containing compositions dissolved in buffered aqueous solutions or pure water for 1:1 w/v solutions, sonicate at an amplitude of from 90 Watts to 720 Watts for from 2-8 minutes duration with 2 second pulses and 2 second pauses. In another embodiment of the present invention, parameters for sonication conditions to provide the most sonicated bioactive lunasin when sonicating lunasin containing compositions dissolved in buffered aqueous solutions or pure water for 2:1 w/v solutions, provide for sonication at an amplitude of from 90 Watts to 810 Watts for from 2-10 minutes duration with 2 second pulses and 2 second pauses. In another embodiment of the present invention, parameters for sonication conditions to provide the most bioactive lunasin when sonicating lunasin containing compositions dissolved in buffered aqueous solutions or pure water for 3:1 w/v solutions, provide for sonication at an amplitude of from 90 Watts to 900 Watts for 2 to 10 minutes duration with 2 second pulses and 2 second pauses.

In other embodiments of the present invention, parameters for sonication conditions to provide increased bioactive lunasin when sonicating lunasin enriched soy powder samples dissolved in buffered aqueous solutions or pure water (1:2 w/v) are contemplated between 10-200 Watts for 0.5-30 minutes duration delivered in approximately 2 seconds pulses with approximately 2 second rests between pulses. For 1:1 w/v solutions, between 20-300 Watts for 1-30 minutes duration delivered in approximately 2 seconds pulses with approximately 2 second rests between pulses. For 2:1 w/v solutions, between 20-400 Watts amplitude and 1.5-30 minutes duration delivered in approximately 2 seconds pulses with approximately 2 second rests between pulses. For 3:1 w/v solutions, between 30-500 Watts and 2-30 minutes duration delivered in approximately 2 seconds pulses with approximately 2 second rests between pulses.

The present invention contemplates that when lunasin is combined with other compositions prior to sonication, the sonication conditions required to obtain improved bioactivity may vary. Preferred sonication parameters can be determined with little experimentation by sampling a few sonication parameters and evaluating the sonicated samples using the screening assays described here and set forth in Example 2.

In certain preferred embodiments of the present invention, lunasin containing compositions to be sonicated according to the methods of the present invention contain an amount of from 0.5 mg to 70 mg of lunasin enriched soy powder per 1 ml of aqueous buffer. In other preferred embodiments of the present invention, lunasin containing compositions to be sonicated according to the methods of the present invention contain an amount of from 1 ug to 7 mg of lunasin per 1 ml of aqueous solution.

Screening Assay

U.S. Pat. No. 9,814,757 teaches an in vitro assay to measure bioactivity of lunasin containing samples. The present invention provides a modified in vitro assay providing higher throughput screening that can be used to screen lunasin and sonicated lunasin for bioactivity to identify material useful in the compositions and methods of the present invention. In one embodiment of the present invention, compositions containing lunasin, or analogues, fragments or variants of lunasin, are screened for bioactivity using the following high throughput screening: around 100 ng of biotin-labeled histone H3 are loaded into each well of a streptavidin-coated microtiter plate and blocked with bovine serum albumin (BSA). Increasing amounts of biotin-labeled acetylated histone H3 are loaded into replicated control wells to generate the standard curve. After washing each well 3× with 200 uL 1×TBS (Tris-buffered saline), sample wells are treated with 5 ug of sonicated LESP mixture in 4 replicates. After a brief wash with 0.5×PBS, the sample wells are treated with a histone acetylase (HAT) reaction mixture containing HAT assay buffer, acetyl-CoA and the PCAF histone acetylase enzyme. After 1 hour shaking incubation at 40° C., samples wells are washed 3× with 1×TBS. Around 100 uL of Anti-acetyl H3 primary antibody (1:5,000 dilution) is added to each well (sample and standard wells) and incubated for 1 hour at RT. Wells are washed 5× with 1×TBS+0.5% Tween 20. Around 100 uL of secondary antibody (goat anti-rabbit IgG at 1:10,000 dilution) is added into each well and incubated for 30 min at room temperature. Plate is washed 3× with 1×TBS+0.5% Tween 20 and 2× with 1×TBS. Around 100 uL of TMB substrate (tetra-methyl-benzidine) is added to each well and incubated for 5-10 minutes for signal detection. Around 50 uL of 1M sulfuric acid is added to each well to stop the reaction and the plate is read on a microplate reader at 450 nm wavelength.

The amount of bioactive lunasin in soy preparations is determined as a percentage of the calculated amount of histone H3 that remains non-acetylated because of the binding and masking effect of lunasin.

Sources of Lunasin.

Naturally-occurring lunasin can be found in significant amounts in soybean seeds and from commercially available sources of soy protein and its analogues from other seed sources such as barley, quinoa, pea and wheat. Because of the biological role of lunasin in the DNA endoreduplication stage of seed development, lunasin and its analogues are expected to be found in the endosperm and cotyledons of other seed-bearing plants (angiosperms) as well. The sonication methods of the present invention can be used on aqueous solutions containing naturally-occurring lunasin from any source, and is not limited to lunasin derived from soy.

Lunasin is found in nature and can be obtained from soybean seeds and from commercially available sources of soy protein and its analogues from other seed sources. For example, without intending to be limited to any method of obtaining lunasin, lunasin can be extracted from the following soy sources: soy flakes, soy flours, soy grits, soy meals, soy protein concentrates, and soy protein isolates, tofu, miso, fermented soy, hydrolyzed soy, and soy milk. The present invention contemplates treatment of any of these sources of lunasin with sonication in liquid to produce sonicated bioactive lunasin. In one aspect of at least one embodiment of the present invention, the lunasin peptides are obtained from soy. In another aspect of at least one embodiment of the present invention, the lunasin peptides are obtained from other seed bearing plants or a combination of soy and other seed bearing plants. Seed bearing plants containing sufficient amounts of lunasin are well known in the art.

U.S. Pat. No. 9,814,757 sets forth detailed information on various sources of lunasin derived from soy, including soy flakes, soy flours, soy grits, soy meals, soy protein concentrates, and soy protein isolates, also referred to as isolated soy proteins, as well as concentration/extraction techniques for lunasin from various sources. These examples are not intended to limit the present invention to any particular source of lunasin or method of concentrating or extracting lunasin.

Administration

Sonicated bioactive lunasin can be administered using a number of different routes including ophthalmic, topical or transdermal, nasal, inhalant, sublingual, buccal, ear, rectal, vaginal, or injection (e.g., intravenously, subcutaneously, intratumorally, intraperitoneally. Administration of compositions for use in the practice of the present invention can be systemic (i.e., administered to the subject as a whole via any of the above routes) or localized (i.e., administered to the specific location of the particular disease or pathological condition of the subject via any of the above routes).

By using sonicated bioactive lunasin in a topical formulation with an appropriate excipient, it is possible to increase the efficacy of lunasin for applications such as reducing skin tumor and cancer formation, actinic keratosis, rosaceae, age and sun spots and other skin diseases associated with abnormal cell division and proliferation. Sonicated compositions containing lunasin can also be used for sublingual, nasal and other routes of delivery that do not expose the composition to digestion, to reduce cholesterol levels and to treat other cholesterol related diseases, such as, without limitation, atherosclerosis, hypertension, obesity and diabetes. Sonicated compositions containing lunasin can also be used to treat or prevent inflammation, atherosclerosis, heart disease, breast cancer, to repair DNA, to protect heart health, to reduce or maintain cholesterol levels, to provide anti-oxidative protection, to promote skin health, to prevent skin aging, to suppress tumor growth, and reduce skin tumor formation, to combat obesity, to treat neurodegenerative disorders like Alzheimer's and chronic traumatic encephalopathy (CTE) and to treat abnormal brain aging.

Dosing

Depending upon the particular needs of the individual subject involved, the compositions of the present invention can be administered in various doses to provide effective treatment concentrations based upon the teachings of the present invention. Factors such as the activity of the selected compositions, the physiological characteristics of the subject, the extent or nature of the subject's disease or pathological condition, and the method of administration will determine what constitutes an effective amount of the selected compositions. Generally, initial doses will be modified to determine the optimum dosage for treatment of the particular subject. Suitable dosages can be chosen by taking into account any or all of such factors as the size, weight, health, age, and sex of the human or individual, the desired goals of the patient, the severity of the pathological condition for which the composition is being administered, the response to treatment, the type and quantity of other medications being given to the patient that might interact with the composition, either potentiating it or inhibiting it, and other pharmacokinetic considerations such as liver and kidney function. These considerations are well known in the art and are described in standard textbooks.

A therapeutically effective amount of any embodiment of the present invention is determined using methods known to pharmacologists and clinicians having ordinary skill in the art. For example, an effective amount can be determined subjectively by administering increasing amounts of the compositions of the present invention until such time the patient being treated shows reduction in cholesterol, total cholesterol, LDL cholesterol or lipid levels. Blood levels of the composition, cholesterol and lipid levels can be determined using routine biological and chemical assays, such as enzyme-linked immunosorbent assay (ELISA), and these blood levels can be matched to the route of administration. The blood level and route of administration giving the most desirable level of cholesterol reduction can then be used to establish an “effective amount” of the pharmaceutical composition for treatment.

This same method of titrating a composition in parallel with administration route can be used to ascertain a therapeutically effective amount of the compositions of the present invention for treating any and all disorders described herein. In addition, animal models as described below can be used to determine applicable dosages for a particular disease or pathological condition. Typically, dosage-effect relationships from in vitro or in vivo tests initially can provide useful guidance on the proper doses for subject administration.

In one exemplary embodiment of the present invention, a product containing an effective amount of sonicated bioactive lunasin is provided. It should be appreciated that the effective amount of sonicated bioactive lunasin will depend, at least in part, on the size, weight, health and desired goals of the individuals being treated with the compositions, and on the condition to be treated or avoided. However, as much of the bioactivity of lunasin is based on its ability to block the acetylation of histone H3 by PCAF, the present invention contemplates the determination of what an effective amount of sonicated bioactive lunasin by quantitatively measuring its ability to block the acetylation of histone H3 by PCAF in vitro.

In one embodiment of the present invention, methods and compositions of the present invention encompass a dose of a composition comprising sonicated bioactive lunasin, or a functionally equivalent variant, analogue or fragment of lunasin, of about 5 ng to about 1000 g, or about 100 ng to about 600 mg, or about 1 mg to about 300 mg, or about 10 mg to about 100 mg. Illustratively, a dosage unit of a composition of the present invention can typically contain, for example, without limitation, about 5 ng, 50 ng 100 ng, 500 ng, 1 mg, 10 mg, 20 mg, 40 mg, 80 mg, 100 mg, 125 mg, 150 mg, 200 mg, 250 mg, 300 mg, 350 mg, 400 mg, 450 mg, 500 mg, 550 mg, 600 mg, 700 mg, 800 mg, 900 mg, 1 g, 5 g, 10 g, 20 g, 30 g, or 40 g of a composition of the present invention.

In certain embodiments of the present invention, a lunasin containing composition at a concentration of from 0.2 mg/ml to 70 mg/ml, and more preferably, between 1 mg/ml and 20 mg/ml, in liquid is treated with sonication. In a preferred embodiment the composition treated with sonication is a combination of soy concentrate and soy flour. In another preferred embodiment, the invention contemplates the sonication of an aqueous solution of a combination of approximately 70% soy concentrate and approximately 30% low fat soy flour at a concentration of between 0.2 mg/ml and 70 mg/ml, more preferably between approximately 1 mg/ml. and 20 mg/ml.

Exemplary dosages for sonicated bioactive lunasin, or fragments, variants and analogues thereof, in accordance with the teachings of the present invention, range from 0.1 ug/ml to 200 mg/ml, preferably, 1 ug/ml to 100 mg/ml, more preferably 10 ug/ml to 50 mg/ml, more preferably 0.1 mg/ml to 10 mg/ml for humans and other individuals having an average weight of 60 kg, although alternative dosages are contemplated as being within the scope of the present invention.

In certain preferred embodiments of the present invention for compositions and methods for administration, sonicated bioactive lunasin, or fragments, variants and analogues thereof, is provided to an individual at a level of between 0.01 mg/Kg and 100 mg/Kg body weight of an individual, preferably 0.05 mg/Kg and 50 mg/Kg, more preferably between 0.5 mg/Kg and 2.5 mg/Kg, and even more preferably between 0.2 mg/Kg and 1.5 mg/Kg.

A dose can be administered in one to about four doses per day, or in as many doses per day to elicit a therapeutic effect. The dosage form can be selected to accommodate the desired frequency of administration used to achieve the specified dosage, as well as the route of delivery.

The amount of therapeutic agent necessary to elicit a therapeutic effect can be experimentally determined based on, for example, the absorption rate of the agent into the blood serum, the bioavailability of the agent, and the ability to inhibit histone H3 acetylation by the histone acetylase enzyme, PCAF. Determination of these parameters is well within the skill of the art.

Formulations.

The invention also concerns formulations containing the compositions of the present invention. The products and compositions of the present invention can be used alone, with other compositions, and/or in carriers for oral, ophthalmic, topical, transdermal, nasal, inhalant, sublingual, buccal, ear, rectal, vaginal, or injection (e.g., intravenously, subcutaneously, intratumorally, intraperitoneally).

In one preferred embodiment the compositions of the present invention are together with a suitable excipient, diluent, or carrier.

The formulations may be a variety of kinds, such as liquid or solid preparations, including sterile injectable solutions, powders, drops, suspensions, syrups, ointments, lotions, creams, pastes, gels, liposomes or the like.

The formulations may be packaged in convenient dosage forms, and may also include other active ingredients, and/or may contain conventional excipients, pharmaceutically acceptable carriers and diluents. The inclusion of the compositions of the present invention in herbal remedies and treatments is also a preferred part of the invention.

Preferred formulations for topical applications of the compositions of the present invention for both pharmaceutical and cosmetic use will employ excipients that are suitable for topical application. Topical formulations typically are gels, salves, powders, or liquids, though controlled formulations which release defined amounts of active ingredient at the desired surface are also desirable. The formulations may contain materials which enhance the permeability of the active moieties through the epidermis. Such penetrants include, for example, DMSO, various bile salts, non-toxic surfactants and the like. Standard ingredients for cosmetic/pharmaceutical compositions are well known in the art, formulations for topical application of pharmaceuticals are found in Remington's Pharmaceutical Sciences, latest edition, Mack Publishing Co., Easton, Pa., incorporated herein by reference. Cosmetic formulations are widely varied and well known to practitioners.

In one preferred embodiment of the present invention, compositions for topical use of the active ingredients are contemplated, whether for strictly cosmetic or pharmaceutical/cosmetic purposes.

Some embodiments of the present invention encompass methods for treating one or more of diseases or conditions for which lunasin is known to treat or prevent or will become known to treat or prevent, comprising treating a patient suffering from one of these diseases or conditions with compositions containing sonicated bioactive lunasin, or functionally equivalent fragments, variants or analogues thereof according to methods of the present invention.

A specific embodiment of the present invention encompasses methods comprising treating, individuals desiring to maintain or obtain a particular level of cholesterol, total cholesterol, LDL cholesterol or lipids with sonicated bioactive lunasin, or functionally equivalent fragments, variants or analogues thereof according to methods of the present invention.

While the primary use of the materials of the invention is intended for humans, there may be instances where treatment is desired on domestic or farm animals or in experimental animals. Indeed, one aspect of the invention is the use of experimental animals to confirm the safety and efficacy of the compositions of the invention. Thus, products intended for use in humans may be applied to laboratory animals such as rats, mice or rabbits to confirm the ability of the individual preparation to treat a particular disease or condition and to assure that an individual preparation is not toxic. The use of the materials of the invention in the context of quality control, as just described, is part of the invention.

It should be understood that the foregoing discussion, embodiments and examples merely present a detailed description of certain preferred embodiments. It will be apparent to those of ordinary skill in the art that various modifications and equivalents can be made without departing from the spirit and scope of the invention.

The following non-limiting examples are provided to better illustrate the present invention. The examples are not intended to limit the scope of the present invention and they should not be so interpreted. Other procedures and adaptations will be apparent to one of ordinary skill in the art upon views these reaction schemes and the structures of the compositions according to the invention. Such procedures are deemed to be within the scope of the present invention. Amounts are in weight parts or weight percentages unless otherwise indicated. All of the cited patents and publications are incorporated herein by reference.

### EXAMPLES

The following examples are provided in order to demonstrate and further illustrate certain preferred embodiments and aspects of the present invention and are not to be construed as limiting the scope thereof. The following experiments were performed to evaluate the effects of sonication on solutions containing lunasin.

### Example 1

Based on the guidance provided in U.S. Pat. No. 9,814,757 to maximize lunasin content of a soy composition, a blend of 70% water washed soy concentrate (ADM, Chicago, Ill.) and 30% low fat soy flour (Harvest Innovations, Indianola, Iowa) was used to make lunasin enriched soy powder (LESP). LESP was dissolved in aqueous buffered solution (1:2 w/v). Approximately 500 mg of LESP was dissolved in 50 ml of 0.1× phosphate-buffered solution (PBS) by stirring and mixing in a 100 ml beaker for 2 hours at room temperature. Stirring rod was removed from the beaker and the mixture was made to undergo sonication using a CGoldenwal™ Ultrasonic Homogenizer and Sonicator (CGoldenwal, Hangzhou, China) with 900 Watt maximum amplitude using a 13 mm ultrasonic probe. Different sonicator settings (time and amplitude) were tested on the LESP mixtures to determine the optimum amount of amplitude (sound energy in Watts) and duration (time in minutes) to yield the most bioactive lunasin. The sonicated mixtures were centrifuged twice at 10,000 G before vacuum filtering to separate insoluble solids from the aqueous layer. The liquid LESP samples were then tested to determine soluble protein concentration using the Bradford protein assay and using bovine serum albumin (BSA) as standard protein controls. Sample aliquots with 200 ug of soluble protein dissolved in 200 uL PBS are transferred to fresh tubes and tested for the presence of sonicated bioactive lunasin using an epigenetic bioassay, as set forth in Example 2.

### Example 2

The following bioassay procedure was performed to determine the amount of bioactive lunasin in sonicated liquid samples of LESP. It can also be used to determine the amount of bioactive lunasin in other lunasin containing compositions.

The standard method for determining lunasin peptide content in soy preparations is by ELISA testing using lunasin antibody for detection and synthetic lunasin as standard control to generate a linear curve for quantitation. However, the lunasin-ELISA method is limited by the fact that lunasin is readily degraded and loses bioactivity when the polypeptide is randomly sheared or broken apart during the process of sonication or when exposed to proteases (enzymes that degrade proteins and polypeptides) found in saliva, blood and epidermal layers of the skin and lungs during sublingual, nasal and topical applications. So even if lunasin is detected by the ELISA method, it does not mean that lunasin is bioactive after sonication and upon sublingual, topical and nasal administrations.

So there is a need for a high-throughput lunasin bioactivity assay that determines how much lunasin is bioactive after undergoing sonication. The bioassay uses the specific binding of lunasin to histone H3 and its inhibition of H3 acetylation by the PCAF histone acetylase enzyme.

Approximately 100 ng of biotin-labeled histone H3 were loaded into each well of a streptavidin-coated microtiter plate and blocked with bovine serum albumin (BSA). Increasing amounts of biotin-labeled acetylated histone H3 were loaded into replicated control wells to generate the standard curve. After washing each well 3× with 200 uL 1× Tris-buffered saline (TBS), sample wells were treated with 5 ug of sonicated LESP mixture in 4 replicates. After a brief wash with 0.5×PBS, the sample wells are treated with a histone acetylase (HAT) reaction mixture containing HAT assay buffer, acetyl-CoA and the PCAF histone acetylase enzyme. After 1 hour shaking incubation at 40° C., samples wells are washed 3× with 1×TBS. Around 100 uL of Anti-acetyl H3 primary antibody (1:5,000 dilution) is added to each well (sample and standard wells) and incubated for 1 hour at room temperature. Wells were washed 5× with 1×TBS+0.5% Tween 20. Around 100 uL of secondary antibody (goat anti-rabbit IgG at 1:10,000 dilution) was added to each well and incubated for 30 min at room temperature. Plate was washed 3× with 1×TBS+0.5% Tween 20 and 2× with 1×TBS. Around 100 uL of TMB substrate (tetra-methyl-benzidine) was added to each well and incubated for 5-10 minutes for signal detection. Around 50 uL of 1 M sulfuric acid was added to each well to stop the reaction and the plate was read on a microplate reader at 450 nm wavelength.

The amount of bioactive lunasin in soy preparations was determined as a percentage of the calculated amount of histone H3 that remains non-acetylated because of the binding and masking effect of lunasin.

Using this lunasin bioassay, we were able to determine which sonication conditions on liquid extracts of lunasin enriched soy powders yielded the most sonicated bioactive lunasin.

The results of the experiment are shown in Table 2, which sets forth the amount of soluble protein and sonicated bioactive lunasin detected from LESP mixtures sonicated under different conditions of amplitude (W) and time (min).

TABLE 2

| | Soluble Protein | | | Amount of Bioactive | | |
|---|---|---|---|---|---|---|
| | Concentration (ug/mL) | | % | Lunasin (ug/mg) | | % |
| Sonication Conditions | Unsonicated | Sonicated | Increase | Unsonicated | Sonicated | Increase |
| 4 min, 2 sec pulse, | 3620.48 | 5092.38 | 40.65 | 36.46 | 56.78 | 55.73 |
| 2 sec off, 360 W | | | | | | |
| 4 min, 2 sec pulse, | 3895.88 | 4430.10 | 13.71 | 33.71 | 39.23 | 16.37 |
| 2 sec off, 270 W | | | | | | |
| 3 min, 2 sec pulse, | 3986.53 | 4213.17 | 5.69 | 39.45 | 34.25 | −13.18 |
| 2 sec off, 450 W | | | | | | |
| 5 min, 2 sec pulse, | 3921.78 | 4012.44 | 2.31 | 40.33 | 36.76 | −8.86 |
| 2 sec off, 360 W | | | | | | |

While several protocols increased bioactivity in the lunasin containing samples, the results show that sonication of 50 ml mixture of 500 mg lunasin enriched soy powder treated for 4 minutes at 360 Watts amplitude with 2 second pulses and 2 seconds off (pause between pulses) showed the most increase in bioactivity of lunasin.

### Example 3

Eight different sample of LESP were treated as set forth in Example 1, using the following sonication conditions, 360 Watts amplitude for 4 minutes performed by 2 second pulses with 2 second pauses in between. Table 3 shows the soluble protein concentration and amount of bioactive lunasin in unsonicated and sonicated samples.

TABLE 3

| | Soluble Protein | | | Amount of Bioactive | | |
|---|---|---|---|---|---|---|
| | Concentration (ug/mL) | | % | Lunasin (ug/mg) | | % |
| Sample Name | Unsonicated | Sonicated | Increase | Unsonicated | Sonicated | Increase |
| Soy Extract B-1 | 3968.33 | 5628.89 | 41.845 | 44.84 | 64.05 | 42.85 |
| Soy Extract B-2 | 3335.40 | 5353.32 | 60.500 | 30.26 | 52.24 | 72.64 |
| Soy Extract B-3 | 3121.03 | 4918.63 | 57.597 | 37.37 | 41.38 | 10.71 |
| Soy Extract B-4 | 3447.70 | 5035.07 | 46.041 | 41.06 | 44.75 | 8.98 |
| Soy Extract B-5 | 3422.18 | 4910.87 | 43.501 | 35.05 | 60.16 | 71.65 |
| Soy Extract B-6 | 3799.89 | 5364.97 | 41.188 | 36.06 | 67.60 | 87.45 |
| Soy Extract B-7 | 3973.43 | 5097.17 | 28.281 | 33.30 | 65.93 | 98.02 |
| Soy Extract B-8 | 3895.88 | 4430.10 | 13.712 | 33.71 | 58.12 | 72.42 |
| Average | 3620.48 | 5092.38 | 40.655 | 36.46 | 56.78 | 55.75 |

Liquid samples of LESP powders have on average 40% higher concentration of soluble proteins detected when sonicated. The breakdown of insoluble protein complexes by sonication increases protein solubility which explains the higher concentrations of soluble protein.

The higher protein concentrations translate to a 55% average increase in amounts of bioactive lunasin in sonicated samples of LESP. This indicates that lunasin bound to these protein complexes have lower bioactivity because of reduced solubility of the lunasin-protein complex and the masking effect of other proteins. Sonication breaks apart these complexes to expose active sites on the lunasin peptide, increase protein solubility and release bioactive lunasin into solution.

A variation in the amount of bioactive lunasin is observed among the different soy extracts. The results in Table 3 show variation in bioactivity in various samples treated by the same protocol. Observation during experimentation revealed that placement of the sonicator probe should be deep enough that it retains contact with the liquid sample throughout sonication, despite any foaming that may occur, in order to maximize efficacy of sonication. The probe in samples B-3 and B-4 of Table 3 were immersed in foam but lost contact with the liquid portion of=the sample, resulting in less improvement in bioactivity.

Although there is an overall increase in bioactive lunasin detected in sonicated liquid samples of LESP powders, it is useful to determine the amount of bioactive lunasin in each sonicated sample using the lunasin bioassay, given the variation in increase in bioactivity seen. This permits one to both maximize increase in bioactivity in the sonicated bioactive lunasin composition and ascertain the proper formulation and standardized dosing of bioactive lunasin in the final product.

The above specification, examples and data provide a complete description of the manufacture and use of the compositions of the invention. While the products, compositions and related methods have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims. All the patents, journal articles and other documents discussed or cited above are herein incorporated by reference.

I claim:

1. A method of increasing the bioactivity of lunasin, comprising: providing a composition comprising lunasin in solution and administering sonication to the composition at an amplitude and time interval that increases the bioactivity of lunasin.

2. The method of claim 1, further comprising, after administering sonication, testing the composition for bioactivity of said lunasin.

3. The method of claim 1, wherein sonication is administered at an amplitude of between 10 Watts and 900 Watts.

4. The method of claim 1, wherein sonication is administered at an amplitude of between 20 Watts and 500 Watts.

5. The method of claim 1, wherein sonication is administered at an amplitude of between 30 and 400 Watts.

6. The method of claim 1, wherein sonication is administered at an amplitude of approximately 360 Watts.

7. The method of claim 1, wherein sonication is administered for a period of between 1 and 20 minutes.

8. The method of claim 1, wherein sonication is administered for a period of between 2 and 10 minutes.

9. The method of claim 1, wherein sonication is administered for a period of approximately 4 minutes.

10. The method of claim 1, wherein sonication is administered in pulses lasting between 1 and 5 seconds.

11. The method of claim 1, wherein the composition comprises lunasin enriched soy powder.

12. The method of claim 1, wherein the composition comprises soy concentrate and soy flour.

13. The method of claim 1, wherein the composition is in solution at a concentration of from 0.5 mg/ml to 70 mg/ml.

14. The method of claim 1, wherein the composition is in solution at a concentration of from 5 mg/ml to 20 mg/ml.

15. The method of claim 1, wherein the composition is in solution at a concentration of approximately 10 mg/mi.

16. The method of claim 1, wherein the composition is in an aqueous solution.

17. A composition comprising sonicated bioactive lunasin.

18. A method of treating a human with lunasin, comprising: (a) providing: (i) an individual desiring or needing treatment with lunasin, and (ii) a composition comprising sonicated bioactive lunasin; and (b) administering said composition to said subject.

19. The method of claim 18 wherein the composition is administered topically.

20. (canceled)

21. (canceled)

22. (canceled)

23. (canceled)

24. (canceled)

25. The method of claim 18 wherein the composition is administered orally.

26. (canceled)

27. (canceled)

28. (canceled)

29. (canceled)

30. (canceled)

31. (canceled)

32. (canceled)

33. (canceled)

34. (canceled)

35. (canceled)


SEQUENCE LISTING (2 sequences)

SEQ ID NO: 1 - protein, 157 aa, Glycine max
MTKFTILLISLLFCIAHTCSASKWQHQQDSCRKQLQGVNLTPCEKHIMEKIQGRGDDDDDDDDDNHILRTMGGRINYIRRNEGKDEDEEEGHMQKCCTEMSELRSPKCQCKALQKIMENQSEELEEKQKKKMEKELINLATMCRFGPMIQCDLSSDD

SEQ ID NO: 2 - protein, 43 aa, Glycine max
SKWQHQQDSCRKQLQGVNLTPCEKHIMEKIQGRGDDDDDDDDD